(19) United States
(12) Patent Application Publication
(10) Pub. No.: US 2025/0172486 A1
(43) Pub. Date: May 29, 2025

(54) METHOD FOR DETECTING, QUANTIFYING AND CHARACTERIZING BRETTANOMYCES SPP YEASTS AND OTHER YEASTS CONTAINED IN AN ORGANIC LIQUID SUBSTRATE CONTAINING FERMENTABLE SUGARS

(71) Applicant: MD INVENTION OENO, Montredon-des-Corbières (FR)

(72) Inventor: Matthieu DUBERNET, NARBONNE (FR)

(73) Assignee: MD INVENTION OENO, Montredon-des-Corbières (FR)

(21) Appl. No.: 18/694,983

(22) PCT Filed: Sep. 27, 2022
(86) PCT No.: PCT/IB2022/059194

§ 371 Date: Mar. 24, 2024

(30) Foreign Application Priority Data
Sep. 28, 2021 (FR) FR2110234

(51) Int. Cl. G01N 15/14; G01N 15/10; G01N 21/64; G01N 33/14

(52) U.S. Cl. CPC G01N 15/1459; G01N 21/6428; G01N 33/143; G01N 33/146; G01N 2015/1006; G01N 2015/1402; G01N 2021/6441; G01N 2333/39

(57) ABSTRACT

The invention relates to a method for detecting, quantifying and differentiating by flow cytometry Brettanomyces spp yeast cells contained in an organic liquid substrate which contains fermentable sugars, whereby a sample of said substrate is taken, optionally diluted, at least a first fluorochrome capable of binding to the DNA of dead and/or live cells is added to said optionally diluted substrate, said sample is irradiated so as to obtain the fluorescence emission of said first fluorochrome and said sample is also irradiated so as to obtain a fluorescence emission of said sample at 670 nm, a biparametric histogram is plotted giving for each point the fluorescence intensity due to the first fluorochrome coupled with the fluorescence intensity emitted at 670 nm, at least a first point cloud is thus obtained corresponding to a greater fluorescence intensity emitted and detected at 670 nm than that detected for the other points, it is inferred therefrom that the points of said first cloud correspond to the Brettanomyces spp cells.

### TECHNICAL FIELD

The present invention relates to a method enabling, with a single sample and a single pass in a cytomer, the detection, quantification and differentiation of Brettanomyces yeasts and other yeasts, in particular Saccharomyces spp contained in an organic liquid substrate containing fermentable sugars.

### PRIOR ART

Brettanomyces genus yeasts are capable of producing compounds that produce, among other things, horse stable odors. Brettanomyces is relatively resistant to alcohol and low pH. In most cases, it is susceptible to SO₂, some strains may be particularly resistant to it. Brettanomyces yeasts are smaller in size compared to Saccharomyces cerevisiae, but they remain difficult to identify on account of substantial polymorphism. Furthermore, in a complex medium such as wine, vinegar, or must, for example, their differentiation is even more complex on account of interactions with the medium.

The most common Brettanomyces yeast is Brettanomyces bruxellensis which comprises a spore-forming genus called Dekkera bruxellensis. These yeasts produce 4-ethyl-phenol in particular, which is stable in wine and gives it a horse stable odor that it is desirable to avoid. The same phenomenon may occur in other fermented and possibly alcoholic juices such as cider, beer, tequila, kombucha, kefir, for example. As Brettanomyces spp is present in the surrounding medium, it is likely to contaminate any liquid substrate containing fermentable sugars in which it can grow.

Brettanomyces yeasts may be presented in several states: live (therefore active and producing the compound cited above), dead (not inactive and not producing the compound cited above) or latent. Latent cells are likely to become active again and thus produce, among other things, 4-ethyl-phenol during wine aging, for example. Cells in the latent state are not detectable by culturing the substrate, which means that culturing a substrate sample does not make it possible to guarantee the absence of undesirable fragrances in the future.

The publication entitled “The application of flow cytometry in microbiological monitoring during winemaking: two case studies” by R. Guzzon & al., published in Ann Microbiol ((2015) 65/1865-1878) states that it is possible to differentiate by flow cytometry a live cell from a dead cell in a pure S. cerevisiae culture or in a wine to which a mixture of sugar and S. cerevisiae has been added. The method applied to wine before the addition of the mixture cited above for a secondary fermentation produces no results. A marker mixture is used in this method: the cFDA mixture combined with propidium iodide.

The publication entitled “Rapid detection of viable yeasts and bacteria in wine by flow cytometry” by Malacrino & al., published in Journal of Microbiological Methods (45 (2001) 127-134) states that it is possible to determine the number of malolactic yeasts and bacteria in Pinot noir juice to which yeasts pre-cultured in a specific medium (1% yeast extract +2% peptone +2% dextrose) have been added by flow cytometry. The best marker is the cFDA fluorescein mixture. The same method applied to a wine sample does not give results consistent with the results obtained by counting on cultures. The wine must therefore be pre-washed before measurement. The publication suggests that this method could be used to detect undesirable yeasts such as Brettanomyces spp.

The publication entitled “Survival and metabolism of hydroxycinnamic acids by Dekkera bruxellensis in monovarietal wines” by Nine de Lima, published in the journal Food microbiology in February 2021 (volume 93), states that it is possible to measure the Dekkera bruxellensis population in wine previously inoculated with a strain of Dekkera bruxellensis by flow cytometry. Two markers were used in combination: propidium iodide and SYTO 9®.

The publication entitled “Specific Identification and Quantification of the Spoilage Microorganism Brettanomyces in Wine by Flow Cytometry: A Useful Tool for Winemakers”, by H. Alexandre & al., published online on Feb. 11, 2010 in the journal Wiley Interscience, describes a method for identifying and quantifying Brettanomyces in wine by flow spectrometry using the fluorescence in situ hybridization method. The fluorescent marker used (Alexa Fluor® 488) targets specific amino acid sequences that correspond to Brettanomyces RNA. The Brettanomyces membrane contained in the wine is first permeabilized, which does not allow differentiation of dead cells from live or latent ones. The detection threshold is 102 cells/mL.

The publication entitled “A simple procedure for detecting Dekkera bruxellensis in wine environment by RNA-FISH using a novel probe” by Branco P & al., published in the international Journal of Food Microbiology Elsevier in 2019, describes a method for detecting D. bruxellensis (Brettanomyces dekkera bruxellensis) in wine using a specific fluorescent probe that targets a specific D. bruxellensis ribosomal RNA sequence. This probe enables good detection if the quantity of ribosomal RNA is substantial enough to obtain a fluorescent signal of sufficient intensity. According to this method, the samples are incubated for 2 hours at 46° C. The red diode (680/30) is used for sample excitation. The cell membranes are permeabilized (duration 1 hour), which does not allow subsequent differentiation of dead cells from live cells.

The publication entitled “Evaluation of damage induced by Kwt and Pikt zymocins against Brettanomyces/Dekkera spoilage yeast, as compared to sulphur dioxide” by Ora L. et al and published in Journal of Applied Microbiology in 2016 suggests using propidium iodide to detect Dekkera bruxellensis. This document concerns the toxicity of two zymocins on Brettanomyces, but does not describe a method for detecting Brettanomyces spp.

The document entitled “Evaluation of yeast viability and concentration during wine fermentation using flow cytometry” by Thornton, p. 209 in the book “Cultivability, mortality and metabolic activity” states that it is possible to detect yeasts in wine and determine whether they are live. However, this document does not describe the possibility of differentiating Brettanomyces spp from other yeasts.

### TECHNICAL PROBLEM

An aim of the present invention is that of providing a flow cytometry method that makes it possible to determine, quantify and differentiate Brettanomyces spp yeasts from other yeasts and in particular from Saccharomyces spp yeasts in a liquid substrate containing fermentable sugars and optionally fine particles of the size of bacteria or yeasts.

A further aim of the present invention is that of providing a flow cytometry method that makes it possible to differentiate and quantify live Brettanomyces spp yeast cells, dead Brettanomyces spp yeast cells and latent Brettanomyces spp yeast cells.

A further aim of the present invention is that of providing a method that is quick to implement and requires in particular a reduced incubation time.

A further aim of the present invention is that of providing a method that is applicable to a finished product, such as wine, cider, beer, vinegar or fruit juice, optionally vatted or bottled and suitable for sale.

A further aim of the invention is that of providing a method that makes it possible, with an analysis of a single sample, to also enumerate bacteria and optionally their state (dead, latent or live)

### BRIEF DESCRIPTION OF THE INVENTION

The present invention relates to a method for detecting, quantifying and differentiating by flow cytometry Brettanomyces spp yeast cells contained in an organic liquid substrate which contains fermentable sugars, whereby a sample of said substrate is taken, optionally diluted, at least a first fluorochrome capable of binding to the DNA of dead and/or live cells is added to said optionally diluted substrate, said sample is irradiated so as to obtain the fluorescence emission of said first fluorochrome and said sample is also irradiated so as to obtain a fluorescence emission of said sample at 670 nm, a biparametric histogram is plotted giving for each point the fluorescence intensity due to the first fluorochrome and the fluorescence intensity emitted at 670 nm, at least a first point cloud is thus obtained corresponding to a greater fluorescence intensity emitted and detected at 670 nm than that detected for the other points, it is inferred therefrom that the points of said first cloud correspond to the Brettanomyces spp cells and the number of Brettanomyces spp cells is optionally enumerated by counting the points of said first cloud.

It is indeed to the Applicant's credit to have demonstrated the autofluorescence of Brettanomyces spp cells at 670 nm after excitation with a laser emitting in the wavelengths corresponding to red (from 620 nm to 750 nm inclusive) and in particular at 637 nm. The first fluorochrome binding to DNA, it makes it possible to suppress background noise due to suspended particles, for example. Only the cells of the microorganisms present, i.e., yeasts, fungi and bacteria, will be labeled. If the first fluorochrome only penetrates cells in which the wall is impaired and therefore permeable (dead cells), the fluorescence signal only makes it possible to detect dead cells, but determining the first window as explained hereinafter and using a second fluorochrome binding to both dead cells and live cells make it possible to detect all Brettanomyces spp cells.

The excitation wavelength is not limited according to the invention. It is advantageously greater than or equal to 620 nm and less than or equal to 750 nm inclusive and in particular equal to 637 nm.

Furthermore, the detection of the autofluorescence of Brettanomyces spp cells makes it possible to define a window quickly and precisely on the biparametric histograms, which imparts a high precision, high reliability and high speed of implementation to the method according to the invention.

Advantageously, the first fluorochrome binds to the DNA of dead and live cells. The sample may be diluted to 1:10, 1:40.1:100, 1:300 or 1;1000, depending on its yeast and bacterial load.

### DETAILED DESCRIPTION

According to a specific embodiment, said substrate contains mostly Brettanomyces spp yeasts and Saccharomyces spp yeasts, at least two point clouds are obtained on said biparametric histogram, one comprising the points corresponding to a greater fluorescence intensity emitted at 670 nm than that of the points of the second point cloud, it is inferred therefrom that the points of said first cloud correspond to the Brettanomyces spp cells and that the points of said second cloud correspond to the Saccharomyces spp cells and the number of Brettanomyces spp and/or Saccharomyces spp cells is optionally enumerated by counting the points of each of said clouds.

Advantageously, according to substrate complexity, before measuring the fluorescence, a first differentiation is carried out between the particles and the cells contained in said sample by measuring the reflected and refracted light intensity and the diffracted light intensity, a biparametric histogram is plotted giving for each point corresponding to a detected particle or cell the value of said intensities, a first window which contains points attributable to yeast cells and optionally a second window which corresponds to points attributable to bacterial cells are thus determined according to the values of said intensities, said biparametric histogram is plotted giving the fluorescence intensity due to the first fluorochrome and the fluorescence intensity emitted at 670 nm for each point located in said first window. Determining the first window (“gating” strategy) makes it possible to reduce the background noise due to particles and bacteria contained in the substrate. The points corresponding to the yeast cells are thus isolated and the biparametric histogram of fluorescence emission cited above is plotted for only these cells. This makes it possible to detect the points actually representing either a bacterium or a particle and still located in the first window.

Advantageously, said first fluorochrome being capable of binding to the DNA of live cells and to the DNA of cells in which the wall is permeable, before any measurement, a second fluorochrome capable of binding only to the DNA of cells in which the wall is permeable is added to said optionally diluted sample, a third window which surrounds the points of said first cloud is determined, the sample is excited in such a way as to induce fluorescence emission of the first and second fluorochrome and, for the points located in said third window, a biparametric histogram is also plotted giving for each point the fluorescence intensity of the first and that of the second fluorochrome or the fluorescence intensity per unit of surface area of one of the two fluorochromes and that due to the other fluorochrome and, furthermore, two groups of points are determined, a first group for which the fluorescence due to the fluorochrome which binds only to the DNA of cells in which the wall is permeable is greater than that of the second group and optionally the number of points of each of the groups is counted, which corresponds to the number of live Brettanomyces spp cells for the second group and the number of dead Brettanomyces spp cells for the first group.

It is also possible to use the fluorescence intensity per unit of surface area for either of the fluorochromes.

The second fluorochrome allows the deletion of points corresponding to particles or bacteria; such points may indeed remain in the first window. The closer the fluorescence emission wavelength of the second fluorochrome is to 670 nm, the greater the fluorescence intensity of the Brettanomyces spp cells labeled using this second fluorochrome. This makes it possible to differentiate dead Brettanomyces spp cells from live Brettanomyces spp cells.

It is also possible to differentiate dead Brettanomyces spp cells using only autofluorescence at 670 nm. A biparametric histogram is then plotted in the third window giving the fluorescence for the first fluorochrome and the fluorescence detected at 670 nm. The fluorescence of dead and/or latent cells at 670 nm is lower than that emitted by live cells.

Advantageously, said second window is determined and said sample is excited in such a way as to induce fluorescence emission of the first and second fluorochrome and, for the points located in said second window, a biparametric histogram is plotted giving for each point the fluorescence intensity of the first and that of the second fluorochrome or the fluorescence intensity per unit of surface area of one of the two fluorochromes and that due to the other fluorochrome and two groups of points are determined, a first group for which the fluorescence due to the fluorochrome which binds only to the DNA of cells in which the wall is permeable is greater than that of the second group and the number of points of each of the groups is counted, which corresponds to the number of live bacterial cells for the second group and the number of dead bacterial cells for the first group.

With a single sample and a single pass in the cytometer, it is possible to obtain a yeast and bacteria analysis simultaneously.

Particularly advantageously, before any measurement, a third fluorochrome, which only emits a fluorescence signal when it reacts with a live cell, is furthermore added to said optionally diluted sample, said sample is excited so as to also obtain the fluorescence emission of said third fluorochrome and for the points of said second and/or third window, a biparametric histogram is plotted giving for each point the fluorescence intensity due to the fluorochrome which only binds to the DNA of cells in which the wall is permeable and the fluorescence intensity due to the third fluorochrome, then for each window, three subgroups of points are determined, a first subgroup of points corresponding to a greater fluorescence intensity due to the third fluorochrome than that of the other subgroups, this first subgroup of points representing live and active Brettanomyces spp/bacterial cells, a second subgroup of points corresponding to a lower fluorescence intensity due to the third fluorochrome than that of the first subgroup and coupled with a lower fluorescence intensity due to the first/second fluorochrome than that of the third subgroup, the points of this second subgroup correspond to Brettanomyces spp cells/bacterial cells in the latent state and a third subgroup of points corresponding to a greater fluorescence intensity due to the first/second fluorochrome than that of the first and second subgroups, these points correspond to dead Brettanomyces spp/bacterial cells.

It is seen here that the presence of the three fluorochromes makes it possible, with a single sample analyzed in one pass in a cytometer, not only to simultaneously detect the presence of yeasts and bacteria, quantify (number of cells) yeasts and bacteria, differentiate Brettanomyces spp yeasts from other yeasts, in particular from Saccharomyces spp, but also determine for bacteria and yeasts, and more specifically for Brettanomyces spp yeasts, their state (dead, live or latent). The method according to the invention even makes it possible to quantify latent yeasts and bacteria, which are not detectable by culture. It also proves to be much more rapid than the method of enumeration after culturing.

The shape of the windows is not limited according to the invention. They may advantageously be polygonal.

According to the invention, the fluorochromes are not limited. Thus, said first and second fluorochromes are different and may be selected from fluorochromes capable of binding to the DNA of cells and having a maximum fluorescence absorption wavelength equal to or greater than 599 nm and equal to or less than 657 nm, a maximum fluorescence emission wavelength equal to or greater than 619 nm and equal to or less than 678 nm and a quantum yield equal to or greater than 0.16 and equal to or less than 0.39 and mixtures thereof, in particular fluorochromes capable of binding to the DNA of cells and having a maximum fluorescence absorption wavelength of 652 nm, a maximum fluorescence emission wavelength of 676 nm and a fluorescence quantum yield on DNA of 0.27 and fluorochromes capable of binding to DNA and having a maximum fluorescence absorption wavelength of 657 nm, a maximum fluorescence emission wavelength of 673 nm and a fluorescence quantum yield on DNA of 0.17, fluorochromes capable of binding only to DNA of cells in which the wall is permeable cell and which have a maximum fluorescence absorption wavelength of 547 nm, a maximum fluorescence emission wavelength of 570 nm and a fluorescence quantum yield on DNA of 0.9 and mixtures thereof.

When said first fluorochrome is selected from fluorochromes capable of binding to DNA and having a maximum fluorescence absorption wavelength of 657 nm, a maximum fluorescence emission wavelength of 673 nm and a fluorescence quantum yield on DNA of 0.17 and fluorochromes capable of binding to the DNA of cells and having a maximum fluorescence absorption wavelength of 652 nm, a maximum fluorescence emission wavelength of 676 nm and a fluorescence quantum yield on DNA of 0.27, the second fluorochrome is selected from fluorochromes capable of binding only to the DNA of cells in which the wall is permeable and which have a maximum fluorescence absorption wavelength of 547 nm and a maximum fluorescence emission wavelength of 570 nm and a fluorescence quantum yield on DNA of 0.9. Said third fluorochrome is advantageously selected from 5-carboxyfluorescein diacetate, 6-carboxyfluorescein diacetate, mixtures of 5-carboxyfluorescein diacetate and 6-carboxyfluorescein diacetate and 5,6 fluorescein carboxylate diacetate succinimidyl ester of the following general formula (1):

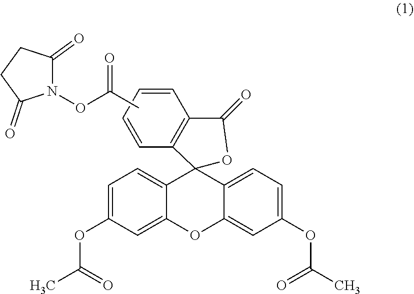

Thus, according to a specific embodiment, said first and said second fluorochromes may be selected from the fluorochromes marketed by Thermofisher under the names SYTOX-orange®, SYTO 62® and SYTO 63®. When said first fluorochrome is selected from SYTO 62® and SYTO (63), the second fluorochrome is SYTOX-orange®. Conversely, when the second fluorochrome is SYTOX-orange, the second fluorochrome is selected from SYTO 62® and SYTO 63® and mixtures thereof. The fluorochrome SYTOX Orange® only penetrates cells in which the cell is impaired, i.e., dead cells. It is preferably used in combination with the third fluorochrome to differentiate the different states of Brettanomyces spp cells (live, latent or dead)

The fluorochrome SYTOX-orange® is marketed by Thermofisher. It is capable of binding to DNA, has a maximum fluorescence absorption wavelength of 547 nm and a maximum fluorescence emission wavelength of 570 nm and a fluorescence quantum yield on DNA of 0.9.

A fluorochrome capable of binding to the DNA of cells and having a maximum fluorescence absorption wavelength of 652 nm and a maximum fluorescence emission wavelength of 676 nm and a fluorescence quantum yield on DNA of 0.27 is marketed under the name SYTO 62® by Thermofisher.

A fluorochrome capable of binding to DNA and having a maximum fluorescence absorption wavelength of 657 nm and a maximum fluorescence emission wavelength of 673 nm and a fluorescence quantum yield on DNA of 0.17 is marketed under the name SYTO 63® by Thermofisher.

The substrate is not limited according to the invention. Thus, the substrate may be selected from optionally sparkling wine, red wine, white wine, rosé wine, cider, beer, sake, fruit juices, in particular grape or apple, water kefir, fruit juice kefir, milk kefir, milk, tequila, whiskey, vodka, must, in particular grape, wines during primary or secondary fermentation, finished wines, optionally sparkling, and vinegars.

The method according to the invention may apply to any Brettanomyces spp yeast species selected from the following species B. anomalus, B. bruxellensis, B. custersianus, B. nanus, B. dekkera bruxellensis and B. naardenensis of at least one other yeast species, and in particular

It makes it possible in particular to differentiate Brettanomyces spp cells from cells of at least one Saccharomyces spp species selected from the following species: Saccharomyces bailii Linder, Saccharomyces carlsbergensis, Saccharomyces cerevisiae, Saccharomyces delbrueckii, Saccharomyces exiguus, Saccharomyces fermentati, Saccharomyces florentinus, Saccharomyces fragilis, Saccharomyces fructuum, Saccharomyces heterogenicus, Saccharomyces oleaginosus, Saccharomyces rosei, Saccharomyces steineri, Saccharomyces boulardii, Saccharomyces kefir, Saccharomyces kluyveri.

The method according to the invention makes it possible in particular to differentiate Brettanomyces dekkera bruxellensis or Brettanomyces bruxellensis from Saccharomyces cerevisiae.

The present invention also relates to a fluorochromic mixture containing or consisting of a solvent and a first fluorochrome selected from fluorochromes capable of binding to DNA and having a maximum fluorescence absorption wavelength of 652 nm and a maximum fluorescence emission wavelength of 676 nm and a fluorescence quantum yield on DNA of 0.27, fluorochromes capable of binding to DNA and having a maximum fluorescence absorption wavelength of 657 nm and a maximum fluorescence emission wavelength of 673 nm and a fluorescence quantum yield on DNA of 0.17 and mixtures thereof, a second fluorochrome selected from fluorochromes having a maximum fluorescence absorption wavelength of 547 nm and a maximum fluorescence emission wavelength of 570 nm and a fluorescence quantum yield on DNA of 0.9 and a third fluorochrome said third fluorochrome is selected from 5-carboxyfluorescein diacetate, 6-carboxyfluorescein diacetate, mixtures of 5-carboxyfluorescein diacetate and 6-carboxyfluorescein diacetate and 5,6 carboxylate fluorescein diacetate succinimidyl ester of the following general formula (1):

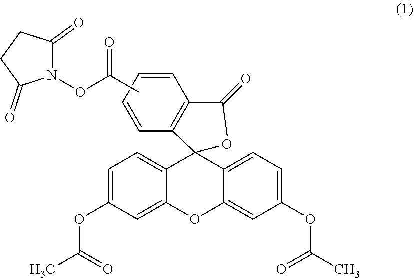

The present invention also relates to the fluorochromic mixture cited above for its use in the in vitro detection (more particularly in a substrate as cited above) of Brettanomyces spp and its differentiation with other yeasts, in particular Saccharomyces spp.

The present invention also relates to the use of the fluorochromic mixture cited above for the detection and differentiation of Brettanomyces spp and its differentiation with other yeasts, in particular Saccharomyces spp, particularly in a substrate as cited above.

According to a specific embodiment, the fluorochromic mixture contains SYTOX®-orange, SYTOR62 or SYTOR 63 and the c-FDA mixture. Similarly, according to an advantageous embodiment, the method according to the invention uses this mixture of three fluorochromes cited above.

The mixture is added to the optionally diluted substrate before its analysis in the flow cytometer.

### DEFINITIONS

The acronym FSC refers to the signal corresponding to diffracted light (FSC: Forward Scatter); this signal depends on the size and surface area of the analyzed cell;

The acronym SSC (SSC: Side Scatter) refers to the signal corresponding to reflected and refracted light; this signal depends on the granularity and cellular complexity of the analyzed cell.

The acronym SSC-H refers to the signal strength corresponding to reflected and refracted light;

The acronym SSC-A refers to the intensity per unit of surface area of the signal corresponding to reflected and refracted light;

The acronyms FSC-H and FSC-A refer, respectively, to the intensity and intensity per unit of surface area of the signal corresponding to diffracted light;

The acronym cFDA or c-FDA denotes a mixture of 5-carboxyfluorescein diacetate and 6-carboxyfluorescein diacetate.

Throughout the application, the maximum fluorescence values in absorption and emission defining the fluorochromes are determined in the presence of DNA with a ratio of about 100 base pairs and in particular 100 base pairs of nucleic acid for a fluorochrome molecule in a Tris medium of pH 7.5 and an EDTA concentration equal to 1 mM.

Throughout the application, the fluorescence quantum yield defining the fluorochromes is measured in the presence of DNA and expressed relative to the yield determined for cresyl violet in methanol.

The term “mostly” means 50% or more by mass or number. When applied to two entities, this means that the mixture of the two entities is present at 50% or more by mass or number.

For the purposes of the present invention, the term “juice” denotes any liquid extracted from the pulp, flesh of certain fruits or vegetables.

For the purposes of the present invention, the term “must” denotes a mixture obtained by pressing or cooking plants (seeds, fruits, leaves, etc.) or plant extracts. These plants may be fruits, vegetables such as potatoes, but also grains such as wheat, barley, malt, maize or rice, for example.

For the purposes of the present invention, the term “wine” denotes a white and/or black grape juice or must of which some or all of the sugar is converted into alcohol by fermentation, in particular by alcoholic fermentation due to Saccharomyces cerevisiae. The wine may be white, red or rose, according to the invention. The wine may be stored in a wooden barrel.

The term “finished wine” denotes a red, white, rosé, optionally sparkling wine, of which the fermentation is complete (primary and secondary in the case of sparkling wines) and which is stored in a vat, wooden barrel or bottle.

The term “beer” denotes any alcoholic beverage obtained by fermentation of a yeast or fungus.

For the purposes of the present invention, the term “vinegar” denotes the result of acetic fermentation produced by microbiological oxidation-reduction of an aqueous ethanol solution, in particular a wine or a cider, exposed to air.

### FIGURES

FIG. 1a is a biparametric histogram representing the intensity of the SSC signal as a function of the intensity of the FSC signal obtained for the analysis of a finished wine sample;

FIG. 1b is a biparametric histogram representing the fluorescence intensity due to the SYTOX-orange® fluorochrome as a function of the fluorescence intensity due to the SYTO 62® fluorochrome for the points located in the “Yeast and Bacteria background” window (first window) shown in FIG. 1a;

FIG. 1c is a biparametric histogram representing the fluorescence intensity due to the cFDA fluorochrome as a function of fluorescence due to the SYTOX-orange® fluorochrome obtained for the “Brettanomyces” window (second window) visible in FIG. 1b;

FIG. 1d is a biparametric histogram giving the fluorescence intensity detected in channel RL1 at a wavelength of 670 nm and the fluorescence intensity detected in channel GL1 at 575 nm;

FIG. 2 represents a biparametric histogram giving for each point the intensity of the SSC signal as a function of the intensity of the FSC signal, the second window corresponding to the bacteria is visible in this figure;

FIG. 3 represents a biparametric histogram giving the intensity per unit of surface area of the SSC signal as a function of the fluorescence intensity of the SYTO-62® fluorochrome for the points located in the “bacteria +background” window (second window) visible in FIG. 2; the window visible in FIG. 3 surrounds the points corresponding to live bacterial cells;

FIG. 4 represents the fluorescence intensity emitted by the SYTOX-orange® fluorochrome as a function of the fluorescence intensity due to the cFDA mixture for the points located in the second window, it represents the three groups of points corresponding to the three states of bacterial cells (live, latent and dead).

### EXAMPLES

### Fluorochrome Labeling Mixture

Physiological saline solution (osmosed/ultra-pure water+NaCl at 7 g/L) is prepared then autoclaved and filtered before use (filter cut-off threshold 0.22 μm).

A fluorochrome marketed under the name SYTO 62™ (SYTO 63™ is also usable) (Thermofisher, 5 μM), SYTOX™-Orange (Thermofisher, 5 μM) and the mixture of 5,6carboxyfluorescein diacetate (c-FDA) are diluted in DMSO (respectively 50 μM, 12.5 μM and 2 g/L in final concentrations) and then stored in a freezer. The final fluorochrome concentrations in the labeling mixture are 0.15UM of SYTO 62™ or 63™, 0.025 μM of SYTOX™-Orange (SYTOX-or) and 2 mg/L of c-FDA in physiological saline solution.

The first fluorochrome has a high affinity for DNA and fluoresces biological organisms containing DNA. It separates microbiological cells from the background noise of the wine. The fluorochrome selected here is SYTOR-63. Its fluorescence is induced by red laser (637 nm). Similar results are also achieved under the same conditions with SYTO® 62.

The second permeating fluorochrome only penetrates cells in which the wall is compromised. The aim is to separate cells with permeable membranes (positively labeled) theoretically corresponding to dead cells, from live cells (unlabeled). The fluorochrome selected here is SYTOX®-Orange in which fluorescence is induced by green laser (532 nm).

The third fluorochrome is an inactive fluorochrome in its initial ester form. It becomes active by esterase activity of cellular metabolisms. The objective is to separate metabolically active cells from those that are not. This second category are populations in latent forms which, in practice, develop little or no growth in cultures on Petri dishes. It corresponds factually to VNC populations (viable non-culturable populations). The fluorochrome selected here is c-FDA. Its fluorescence is induced by blue laser (488 nm).

### Substrate Sample Preparation

### Wine and Must Samples

Samples of finished or fermenting (must) wines are diluted (to 1:40, 1:100, 1:300 or 1:1000 depending on their bioburden) with the fluorochromic labeling mixture. For packaged wines, 50 mL is centrifuged for 8 min at 4500 rpm. The supernatant is removed and the pellet is taken up in 10 mL of filtered physiological saline solution (8.5 g/L of NaCl in osmosed water and filtered at 0.22 μm). A 1:2 dilution of the sample in the labeling mixture is produced, then the whole is vortexed for a few seconds. The mixture of substrate+fluorochromic mixture is incubated for about 30 min protected from light before analysis.

The sample is a sample of 2020 finished wine from the Languedoc region.

### Materials Used

Cytometer: ATTUNER NXT acoustic focusing cytometer (thermofisher scientific). This method describes a microbiological analysis protocol using a flow cytometer equipped with 3 lasers: blue (488 nm), green (532 nm) and red (637 nm). Triple cell labeling of bacteria and yeasts using the fluorochromes cited above is performed. The cytometer is optionally equipped with an automatic sample changer for reading 96-well microplates. This equipment is equipped with an acoustic flow focusing system making it possible to use a flow rate of up to 1000 μL/min.

The flow rate is set to 500 μL/min. The latter is slowed down when the bioburden is high. The data are collected on the following channels: FSC, SSC, BL1 (525/50) for c-FDA, GL1 (575/36) for SYTOX-orange and RL1 (670/14) for SYTO 62 or 63. The voltage values for each of these channels are 200V, 300V, 330V, 360V and 440V respectively. Different values may be applied. There is no compensation problem to correct in this configuration.

### Processing of Results

Events responding to SYTO 6255® or 63-positive (SYTO 62 or 63+) are considered as microorganisms.

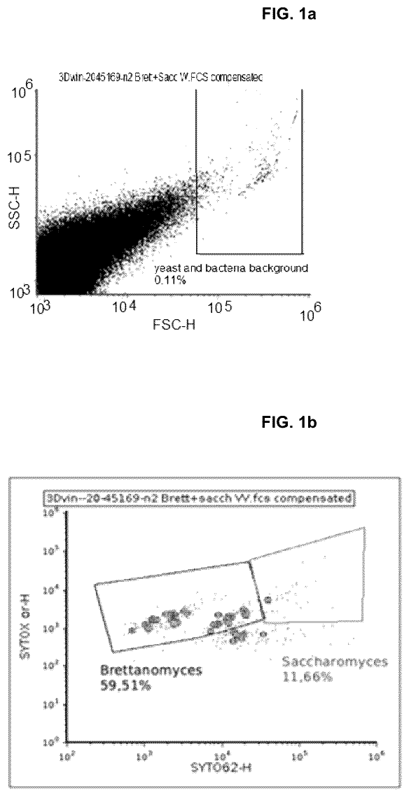

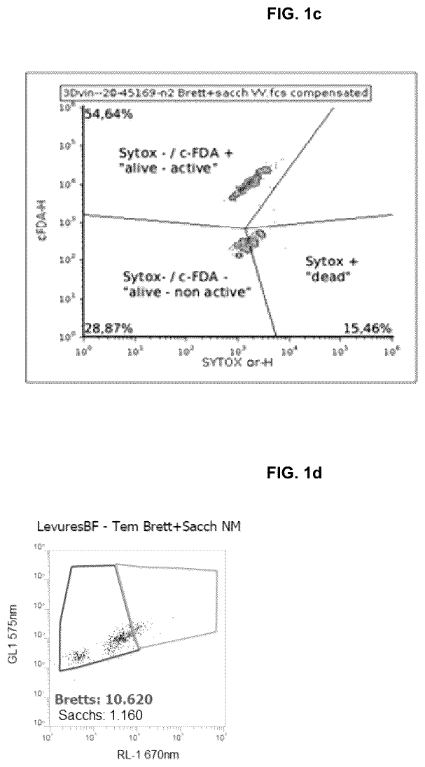

Triple sample labeling and a calibration strategy allowed the separation of bacteria, Saccharomyces and Brettanomyces yeasts in the finished wine. Initially, most of the bacteria and the background noise are separated from the yeasts and some bacteria by the SSC-H/FSC-H plot (FIGS. 1a and 2). Then, thanks to the RL1 plot (670/14 nm) applied to the yeast-bacteria window of FIG. 1a, the last bacteria are removed and the dichotomy between Brettanomyces and Saccharomyces appears. This dichotomy is enabled thanks to a manifest property of different relative levels of autofluorescence in red of Saccharomyces spp and Brettanomyces spp cells.

As shown in FIG. 1d, even in the absence of SYTO62/63 labeling, the difference in fluorescence at 670 nm between Saccharomyces and Brettanomyces can be observed (see FIG. 1d). This is therefore a differentiated autofluorescence phenomenon.

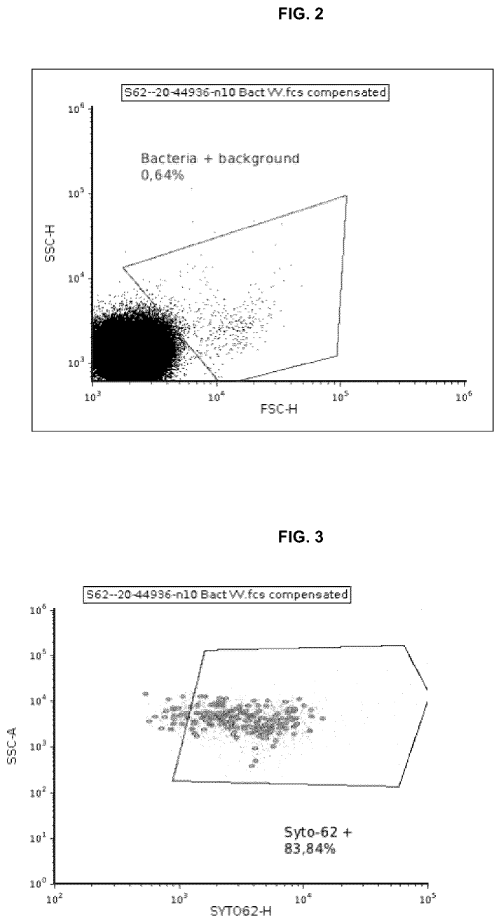

FIG. 3 shows the difference in fluorescence between dead bacterial cells and latent or live ones. FIG. 1b shows this difference for bacteria.

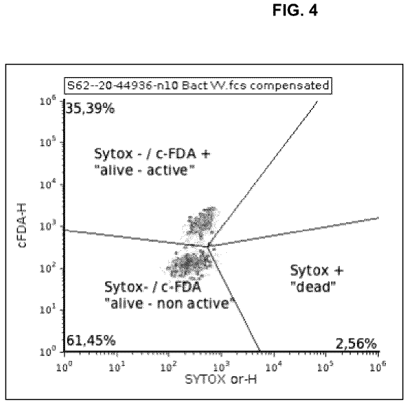

Then, the different states of Brettanomyces and bacteria are obtained by cross-checking the data obtained by the 3 fluorochromes. Thus, the “dead” state corresponds to SYTOX-Orange +/cFDA−, the “live latent (VNC)” state to SYTOX-Orange−/c-FDA−, and the “live active/vital” state to SYTOX-Orange−/c-FDA+ (see FIG. 1c and FIG. 4). It was possible to validate the windows by the work comparing cytometry with microscopy and Petri dishes.

In the light of the results cited above, it is noted that the method according to the invention makes it possible to, simultaneously, separate the microorganism of interest from the background noise, separate live microorganisms from dead microorganisms and within the live microorganism population, separate physiologically active microorganisms from dormant microorganisms. It simultaneously provides all the vitality and viability information. It can be implemented quickly with active labeling in about 15 minutes. It is also less expensive because it uses existing reagents. It can be industrialized for high-throughput analysis.

Of course, the invention is described above by way of example. It is understood that a person skilled in the art is capable of creating various alternative embodiments of the invention without for all that leaving the scope of the invention.

1. A method for detecting, quantifying and differentiating by flow cytometry Brettanomyces spp yeast cells contained in an organic liquid substrate which contains fermentable sugars, whereby a sample of the substrate is taken, optionally diluted, at least a first fluorochrome capable of binding to the DNA of dead and/or live cells is added to the optionally diluted substrate, the sample is irradiated so as to obtain the fluorescence emission of the first fluorochrome and the sample is also irradiated so as to obtain a fluorescence emission of the sample at 670 nm, a biparametric histogram is plotted giving for each point the fluorescence intensity due to the first fluorochrome and the fluorescence intensity emitted at 670 nm, at least a first point cloud is thus-obtained corresponding to a greater fluorescence intensity emitted and detected at 670 nm than that detected for the other points, wherein the points of the first cloud correspond to the Brettanomyces spp cells and the number of Brettanomyces spp cells is optionally enumerated by counting the points of the first cloud.

2. The method of claim 1, characterized in that the substrate contains mostly Brettanomyces spp yeasts and Saccharomyces spp yeasts, in that two point clouds are obtained on the biparametric histogram, a first cloud comprising the points corresponding to a greater fluorescence intensity emitted at 670 nm than that of the points of the second point cloud, wherein the points of the first cloud correspond to the Brettanomyces spp cells and that the points of the second cloud correspond to the Saccharomyces spp cells and the number of Brettanomyces spp and/or Saccharomyces spp cells is optionally enumerated by counting the points of each of the clouds.

3. The method of claim 1, characterized in that before measuring the fluorescence, a first differentiation is carried out between the particles and the cells contained in the sample by measuring the reflected and refracted light intensity and the diffracted light intensity, a biparametric histogram is plotted giving for each point corresponding to a detected particle or cell the value of the intensities, a first window which contains points attributable to yeast cells and optionally a second window which corresponds to points attributable to bacterial cells are thus-determined according to the values of the intensities, the biparametric histogram is plotted giving the fluorescence intensity due to the first fluorochrome and the fluorescence intensity emitted at 670 nm for each point located in the first window.

4. The method of claim 1, characterized in that the first fluorochrome being capable of binding to the DNA of live cells and to the DNA of cells in which the wall is permeable, in that before any measurement, a second fluorochrome capable of binding only to the DNA of cells in which the wall is permeable is added to the optionally diluted sample, in that a third window that surrounds the points of the first cloud is furthermore determined, in that the sample is excited in such a way as to induce fluorescence emission of the first and second fluorochrome and, for the points located in the third window, a biparametric histogram is also plotted giving for each point the fluorescence intensity of the first and that of the second fluorochrome or the fluorescence intensity per unit of surface area of one of the two fluorochromes and that due to the other fluorochrome and in that, furthermore, two groups of points are determined, a first group for which the fluorescence due to the fluorochrome which binds only to the DNA of cells in which the wall is permeable is greater than that of the second group and the number of points of each of the groups is counted, which corresponds to the number of live Brettanomyces spp cells for the second group and the number of dead Brettanomyces spp cells for the first group.

5. The method of claim 2, characterized in that the second window is determined and in that the sample is also excited in such a way as to induce fluorescence emission of the first and second fluorochrome and, for the points located in the second window, a biparametric histogram is plotted giving for each point the fluorescence intensity of the first and that of the second fluorochrome or the fluorescence intensity per unit of surface area of one of the two fluorochromes and that due to the other fluorochrome and in that two groups of points are determined, a first group for which the fluorescence due to the fluorochrome which binds only to the DNA of cells in which the wall is permeable is greater than that of the second group and the number of points of each of the groups is counted, which corresponds to the number of live bacterial cells for the second group and the number of dead bacterial cells for the first group.

6. The method of claim 5, characterized in that before any measurement, a third fluorochrome, which only emits a fluorescence signal when it reacts with a live cell, is furthermore added to the optionally diluted sample, the sample is excited so as to also obtain the fluorescence emission of the third fluorochrome and for the points of the second and/or third window, a biparametric histogram is plotted giving for each point the fluorescence intensity due to the fluorochrome which only binds to the DNA of cells in which the wall is permeable and the fluorescence intensity due to the third fluorochrome, then for each window, three subgroups of points are determined, a first subgroup of points corresponding to a greater fluorescence intensity due to the third fluorochrome than that of the other subgroups, this first subgroup of points representing live and active Brettanomyces spp/bacterial cells, a second subgroup of points corresponding to a lower fluorescence intensity due to the third fluorochrome than that of the first subgroup and coupled with a lower fluorescence intensity due to the first/second fluorochrome than that of the third subgroup, the points of this second subgroup correspond to Brettanomyces spp cells/bacterial cells in the latent state and a third subgroup of points corresponding to a greater fluorescence intensity due to the first/second fluorochrome than that of the first and second subgroups, these points correspond to dead Brettanomyces spp/bacterial cells.

7. The method of claim 1, characterized in that the first and second fluorochromes are different and selected from fluorochromes capable of binding to the DNA of cells and having a maximum fluorescence absorption wavelength equal to or greater than 599 nm and equal to or less than 657 nm, a maximum fluorescence emission wavelength equal to or greater than 619 nm and equal to or less than 678 nm and a quantum yield equal to or greater than 0.16 and equal to or less than 0.39 and mixtures thereof, in particular fluorochromes capable of binding to the DNA of cells and having a maximum fluorescence absorption wavelength of 652 nm, a maximum fluorescence emission wavelength of 676 nm and a fluorescence quantum yield on DNA of 0.27 and fluorochromes capable of binding to DNA and having a maximum fluorescence absorption wavelength of 657 nm, a maximum fluorescence emission wavelength of 673 nm and a fluorescence quantum yield on DNA of 0.17, fluorochromes capable of binding only to the DNA of cells in which the wall is permeable and which have a maximum fluorescence absorption wavelength of 547 nm, a maximum fluorescence emission wavelength of 570 nm and a fluorescence quantum yield on DNA of 0.9 and mixtures thereof, and in that when the first fluorochrome is selected from fluorochromes capable of binding to DNA and having a maximum fluorescence absorption wavelength of 657 nm, a maximum fluorescence emission wavelength of 673 nm and a fluorescence quantum yield on DNA of 0.17 and fluorochromes capable of binding to the DNA of cells and having a maximum fluorescence absorption wavelength of 652 nm, a maximum fluorescence emission wavelength of 676 nm and a fluorescence quantum yield on DNA of 0.27, the second fluorochrome is selected from fluorochromes capable of binding only to the DNA of cells in which wall is permeable and which have a maximum fluorescence absorption wavelength of 547 nm and a maximum fluorescence emission wavelength of 570 nm and a fluorescence quantum yield on DNA of 0.9 and in that the third fluorochrome is selected from 5-carboxyfluorescein diacetate, 6-carboxyfluorescein diacetate, mixtures of 5-carboxyfluorescein diacetate and 6-carboxyfluorescein diacetate and 5,6 carboxylate fluorescein diacetate succinimidyl ester of the following general formula (1):

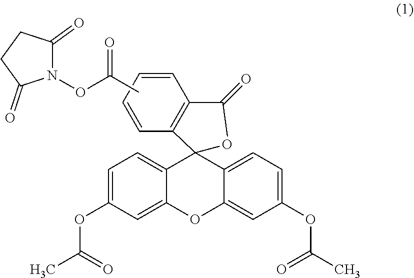

8. The method of claim 1, characterized in that the substrate is selected from optionally sparkling wine, red wine, white wine, rosé wine, cider, beer, sake, fruit juices, in particular grape or apple, water kefir, fruit juice kefir, milk kefir, milk, tequila, whiskey, vodka, must, in particular grape, wines during primary or secondary fermentation, finished wines, optionally sparkling, and vinegars.

9. The method of claim 1, characterized in that it makes it possible to detect, quantify and differentiate at least one Brettanomyces spp yeast species selected from the following species B. anomalus, B. bruxellensis, B. custersianus, B. nanus, B. dekkera bruxellensis and B. naardenensis from at least one other yeast species and in particular from at least one Saccharomyces spp species selected from the following species: Saccharomyces bailii Linder, Saccharomyces carlsbergensis, Saccharomyces cerevisiae, Saccharomyces delbrueckii, Saccharomyces exiguus, Saccharomyces fermentati, Saccharomyces florentinus, Saccharomyces fragilis, Saccharomyces fructuum, Saccharomyces heterogenicus, Saccharomyces oleaginosus, Saccharomyces rosei, Saccharomyces steineri, Saccharomyces boulardii, Saccharomyces kefir, Saccharomyces kluyveri and in particular Saccharomyces cerevisiae.

10. The method of claim 1, characterized in that the sample is excited at a wavelength greater than or equal to 620 nm and less than or equal to 750 nm inclusive and in particular equal to 637 nm.

11. A fluorochromic mixture containing or consisting of a solvent and a first fluorochrome selected from fluorochromes capable of binding to DNA and having a maximum fluorescence absorption wavelength of 652 nm and a maximum fluorescence emission wavelength of 676 nm and a fluorescence quantum yield on DNA of 0.27, fluorochromes capable of binding to DNA and having a maximum fluorescence absorption wavelength of 657 nm and a maximum fluorescence emission wavelength of 673 nm and a fluorescence quantum yield on DNA of 0.17 and mixtures thereof, a second fluorochrome selected from fluorochromes having a maximum fluorescence absorption wavelength of 547 nm and a maximum fluorescence emission wavelength of 570 nm and a fluorescence quantum yield on DNA of 0.9 and a third fluorochrome the third fluorochrome is selected from 5-carboxyfluorescein diacetate, 6-carboxyfluorescein diacetate, mixtures of 5-carboxyfluorescein diacetate and 6-carboxyfluorescein diacetate and 5,6 carboxylate fluorescein diacetate succinimidyl ester of the following general formula (1): [chemical structure]
